(19) United States
(12) Patent Application Publication
(10) Pub. No.: US 2018/0057879 A1
(43) Pub. Date: Mar. 1, 2018

(54) DIAGNOSTICS FOR DETERMINING THE ONSET OF ALZHEIMER'S DISEASE

(71) Applicant: MACCONNELL RESEARCH CORPORATION, San Diego, CA (US)

(72) Inventors: William P. MacConnell, Solana Beach, CA (US); Harold Amogan, Imperial Beach, CA (US)

(21) Appl. No.: 15/252,531

(22) Filed: Aug. 31, 2016

(51) Int. Cl. C12Q 1/68

(52) U.S. Cl. CPC C12Q 1/6883; C12Q 1/6888

(57) ABSTRACT

In accordance with the present invention, a methodology for diagnosing the onset of a neurodegenerative disease, such as Alzheimer's disease, is provided. For this methodology, predetermined sequences of DNA fragments from a microbial nucleic acid are obtained from the Cerebral Spinal Fluid (CSF) of a patient and identified. The DNA fragments are then compared with a database of genomic microbial sequences. The detection of similarities between the DNA fragments from the CSF, and the data recorded in the database, can then be used to determine the extent of a microbial infestation involving a neurodegenerative disease such as Alzheimer's disease.

### FIELD OF THE INVENTION

The present invention pertains generally to methodologies and means for implementing the methodologies for diagnosing a microbial infection. More particularly, the present invention pertains to methodologies wherein microbial nucleic acid from a sample of Cerebral Spinal Fluid (CSF) is sequenced to identify DNA fragments of nucleic acid. The present invention is particularly, but not exclusively, useful as a methodology wherein sequences of DNA fragments from CSF are compared with a database to identify the onset of a neurodegenerative disease such as Alzheimer's disease.

### BACKGROUND OF THE INVENTION

Alzheimer's disease (AD) is a neurodegenerative disease that is presently not detectable until the advanced, late stages of the disease. Unfortunately, in these late stages, there is no truly effective treatment for the disease. To date, approximately 5 million individuals in the U.S. suffer from Alzheimer's disease, with approximately 16,000 deaths attributed to AD per year. A fortunate consequence of this is that much effort has been made to advance and encourage research for both a diagnosis and a treatment for AD, as well as many other neurodegenerative diseases. Despite this, continuing questions have been, and remain; what is the cause of these diseases, and how can they be properly diagnosed.

Recent research, however, has detected a microbial infection that is present in the brains of elderly patients who have died of AD. Research has also shown that a same, or similar, microbial population is also present in the Cerebral Spinal Fluid (CSF) of each of these AD patients.

To demonstrate this correlation, below is a table of actual microbial identification that have been determined from seven autopsied human brain samples and their corresponding CSF samples.

### Sample of Bacterial Strains Identified in Autopsied Alzheimer's Brain Tissue Samples and Corresponding CSF Samples using Amplification and DNA Sequence Identification

Data from Brain samples are indicated in Bold type—Data from CSF samples are bracketed { }.

TABLE

| Patient | | | Stenotro- | Propioni- | |
|---|---|---|---|---|---|
| ID | Age | Delftia | phomonas | bacterium | Other |
| 4279 | 76 | 4, {9} | 2, {2} | | |
| 4312 | 78 | {18} | | | [symbol] |
| 4170 | 64 | 5, {9} | 2 | 3, {1} | [symbol] |
| | | | | | [symbol] |
| | | | | | {1 Streptococcus} |
| 4202 | 39 | 6, {6} | 2, {2} | | [symbol] |
| 4203 | 72 | 5, {2} | | | |
| 4130 | 67 | {1} | 1, {2} | | {3 Streptococcus} |
| 4340 | 47 | 40, {1} | 22, {1} | 3 | [symbol] |
| | | | | | [symbol] |
| | | | | | [symbol] |

For diagnostic purposes, the above factors are important considerations for isolating and identifying microbial nucleic acids in the CSF of a patient that are responsible for AD (or for other specified neurodegenerative diseases). The impetus here is that an ability to diagnose AD, before it has advanced to a point where treatment may not be possible, is very important.

Fortunately, there are many well-known techniques and procedures that are presently available to analyze and evaluate genomic DNA and RNA. In particular, it is possible to isolate DNA fragments of nucleic acid from a microbial genome to determine the extent of a microbial infestation. The present invention envisions that this can be done for microbial genomes that are responsible for AD.

In light of the above, it is an object of the present invention to provide a method for assaying microbes in the Cerebral Spinal Fluid (CSF) of a living patient to detect the onset of a neurodegenerative disease. Another object of the present invention is to provide a diagnostic test for the early detection of AD. Yet another object of the present invention is to provide a diagnostic test for AD that is easy to implement, reliable and relatively cost effective.

### SUMMARY OF THE INVENTION

In accordance with the present invention, a method for assaying microbes in the Cerebral Spinal Fluid (CSF) of a patient is provided for the purpose of detecting an onset of a neurodegenerative disease. In particular, the neurodegenerative disease of interest for the present invention is Alzheimer's disease (AD). It is an important consideration that the present invention is based on research data which indicates, and suggests, that AD and other neurodegenerative diseases may be caused by microbial infections in the brain. Moreover, as envisioned for the present invention, these infections can be diagnosed by analyzing and evaluating microbial DNA samples obtained from the Cerebral Spinal Fluid of a patient. Importantly, such diagnoses can be made well before other known symptoms of the disease become apparent.

The methodology of the present invention begins by obtaining a biopsy of CSF from a patient. A sample of microbial nucleic acid (i.e. microbial DNA) in the CSF is then purified from the biopsied sample and prepared for clinical evaluation. Specifically, this preparation requires releasing the nucleic acid from the microbial and host cells in the CSF. A well-known procedure for doing this is a so-called “Bead Blast” procedure. In particular, this procedure requires oscillating the CSF sample in a vial containing beads along with lysis chemicals and enzymes (such as proteases). The result here is that collisions between the beads and the host/microbial cells in the vial will release microbial nucleic acid from these microbes and cells in the CSF sample. The released microbial nucleic acid is then purified by well-known procedures.

After the microbial nucleic acid from the CSF has been purified, it needs to he amplified. This can be done by means well-known in the pertinent art, such as Polymerase Chain Reaction (PCR) amplification techniques. For the present invention, however, the amplification requires first determining a nucleic acid sequence of interest (i.e. a target sequence). In particular, the target sequence is predetermined, and it will include conserved regions in a fragment of the microbial genome of interest.

Of particular concern for the present invention are the conserved regions in a fragment of the nucleic acid having a sequence that includes 16S, 23S, and internal Transcribed Sequence (ITS) ribosomal regions of the microbial genome. Once such a nucleic acid sequence has been determined, a plurality of different oligonucleotide primers is selected for amplifying respective regions of the microbial genome using PCR. In an example of this type of amplification, a 1492 forward primer recognizes its respective 16S ribosomal region of the microbial genome, and a 457 reverse primer recognizes its respective 23S ribosomal region of the microbial genome. Accordingly, a specific, predetermined portion (i.e. fragment) of nucleic acid is amplified.

The amplified microbial nucleic acid is then manipulated to detect and identify the extent of microbial infestation present in the CSF biopsy. According to the present invention, this manipulation can be accomplished in either of several different ways. One way is by directly sequencing the amplified fragments to identify the presence of the target nucleic acid sequence. Another is by cloning amplified fragments into a plasmid replication vector to create molecular clones, and then sequencing the molecular clones to identify the presence of the target nucleic acid sequence. In each case, the determined nucleic acid sequence can then be compared with a database to identify the extent of microbial infestation present in the CSF biopsy.

As an alternative to the sequencing procedures disclosed above, amplified fragments can be strand denatured then hybridized to an array of separated oligonucleotides that are bound to a hybridization membrane. The array-bound amplified fragments can then be probed to detect positive hybridization of specific sequences which will allow for the evaluation of microbial presence in the original CSF sample. In yet another alternative procedure, a sample of the unpurified CSF biopsy can be spread on different agar medium growth plates. The presence or absence of varying growth rates for the bacteria contained in the CSF sample on each of the different plates can then be observed, to thereby identify a source of the microbial infestation.

It is also envisioned for the present invention that a complete diagnostic procedure can be periodically accomplished. For example, a testing procedure can be repeated at predetermined time intervals, such as every two years. Also, the present invention envisions that microbes of interest will include bacteria, protozoa, fungi and archaea. As noted above, the neurodegenerative disease of primary interest for the present invention is Alzheimer's disease. As intended for the present invention, the nucleic acid is DNA, or it can be DNA that is created by reverse transcription of RNA.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a systematic presentation of tasks in a methodology for the present invention shown together with an assembly of instruments and equipment (i.e. an implementer) which can be employed to perform tasks of the methodology for an implementation of the methodology; and

FIG. 2 is an exemplary microbial genomic DNA fragment showing regions of interest for the diagnosis of Alzheimer's disease.

### DESCRIPTION OF THE PREFERRED EMBODIMENTS

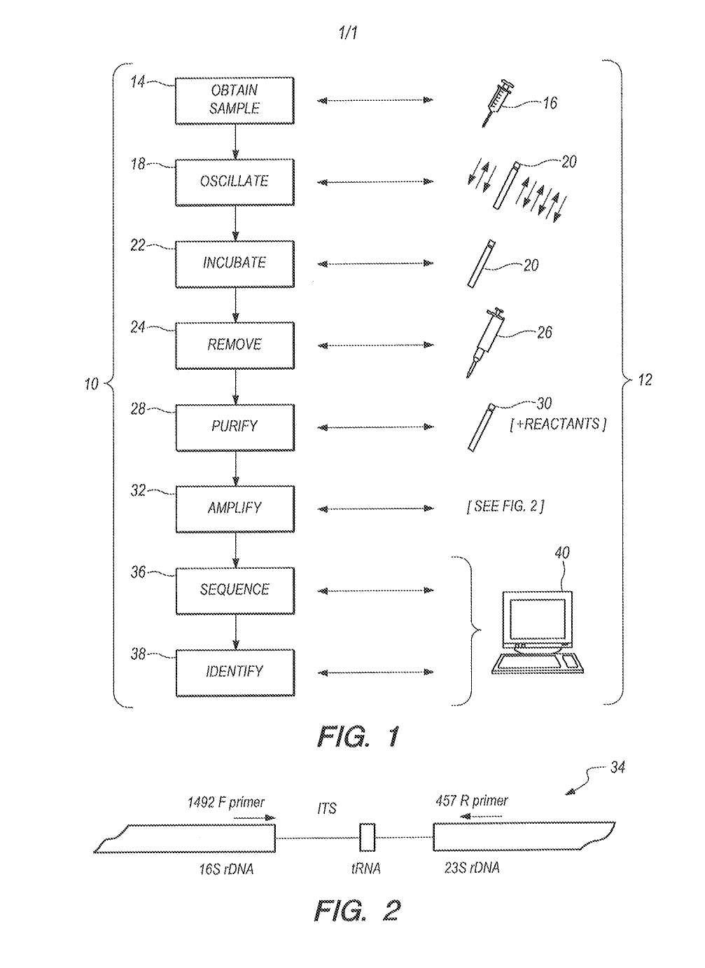

Referring initially to FIG. 1, a methodology for the present invention is shown and is designated 10. In addition to the methodology 10, FIG. 1 also shows an implementer 12, which is essentially an assembly or collection of different instruments, machines, protocols, components, devices and computers/comparators that may be employed to perform the methodology 10 of the present invention. Where specifically applicable, items for the implementer 12 are disclosed and numerically characterized below.

As indicated by block 14 in FIG. 1, the methodology 10 requires first obtaining, a sample of Cerebral Spinal Fluid (CSF) from a patient (not shown). This is accomplished using a syringe 16 of a type well-known in the pertinent art for the particular purpose. Block 18 indicates that the obtained CSF sample is then placed in a vial 20 along with beads and lysis buffers and oscillated. Preferably, this oscillation is accomplished by a device (not shown) that can perform a so-called Bead Blast procedure in which nucleic acids are released from various known and unknown microbial species that may be present in the CSF sample.

A typical Bead Blast procedure begins by placing a sample of CSF into a 1.5-2.0 ml crew cap vial containing approximately 1 mm diameter ceramic, glass, or zirconium beads. A sufficient amount of lysis buffer is added to the vial 20 to submerse or dilute the starting sample that is presumed to contain microbes and host tissue or cells. Lysis buffer typically contains 60 mM Tris pH 8.0, 10 mM EDTA, 0.2% sodium dodecyl sulfate (SDS), and a 0.2 milligrams/milliliter of protease, such as proteinase K. The sealed screw-cap vial 20 is then rapidly oscillated for a period of 0.5 to 3 minutes. The oscillating speed is typically 700-4000 rpm. During this process, the beads collide with each other, which affects the breaking open of virtually any type of microbial or host cell, and thus releases their internal nucleic acids into the lysis solution. The presence of the EDTA, SDS detergent, and protease further renders the nucleic acid in soluble form by digesting and removing proteins and other molecules bound to the nucleic acid.

After the Bead Blast procedure has been completed, block 22 indicates that the vial 20 containing lysed cells is incubated for around 30-190 minutes at 40-65° C. The incubation procedure may be abbreviated or omitted in some cases. Block 24 then indicates that after completion of the incubation period, a solubilized nucleic acid (i.e. lysed cells) is removed from the vial 20 by a pipette 26 and purified (see block 28). This is done in another vial 30. In accordance with the present invention this purification can be accomplished by any proper protocol (procedure) that is well-known in the pertinent art, such as phenol/chloroform extraction followed by ethanol/salt precipitation, or by use of commercially available kits or other reagents. After the CSF sample has been purified (see block 28), it is to be amplified, block 32.

The amplification of a purified CSF sample, as indicated by block 32 for the present invention, can be accomplished using Polymerase Chain Reaction (PCR) techniques for DNA, or reverse transcription-PCR in the case of RNA. In either case, however, it is important that a genomic DNA fragment such as shown in FIG. 2, and generally designated 34, will be specifically targeted. To do this, oligonucleotide primers that are known to recognize conserved sequence regions of a DNA fragment 34 can be used in PCR. Examples of universal primers include: the 16S: 1492 forward primer (5′ AAG TCG TAA CAA GGT AAC CG-3′) and the 23S: 457 reverse primer (5′-CCT TTC CCT CAC GGT ACT-3′). When used together in PCR, these primers generate DNA fragments beginning at the very end of the 16S ribosomal DNA region, continuing through the ITS region, and containing the first 457 bases of the 23S RNA. PCR amplification of this sort using CSF purified genomic DNA typically produces a mixture of 700-1400 base pair fragments of DNA, with fragment length depending on the species of bacteria contained in the sample. The DNA sequence of these amplified DNA bands act as a fingerprint to positively identify the various species of bacteria that were present in the original sample.

After the ribosomal DNA amplification indicated by block 32 has been accomplished, for a preferred embodiment of the present invention, block 36 indicates that amplified fragments 34 need to be sequenced. Block 38 further indicates the sequenced fragments 34 need to be identified. Preferably, the identification tasks in the methodology 10 are accomplished together using a computer 40. Moreover, research tools such as Next Generation Sequencing (NGS) can be used to directly determine the DNA sequence of multiple fragments of PCR amplified microbial DNA, without the need for cloning. Additionally, the present invention recognizes that as alternatives to sequencing techniques, other techniques may also be used to identify the extent of microbial infestation present in the CSF biopsy in accordance with the present invention. For example: i) employing an array of separated oligonucleotides bound to a hybridization membrane for detection probing; or ii) using a plurality of agar medium growth plates to monitor microbial growth activity of the unpurified CSF sample can be employed.

While the particular Diagnostics for Determining the Onset of Alzheimer's Disease as herein shown and disclosed in detail is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A method for assaying microbes in the Cerebral Spinal Fluid (CSF) of a patient to detect an onset of a neurodegenerative disease which comprises the steps of:
obtaining a biopsy of CSF containing a microbial nucleic acid;
releasing the microbial nucleic acid from host/microbial cells in the CSF;
purifying the microbial nucleic acid from the CSF;
amplifying a predetermined fragment of the purified nucleic acid; and
manipulating the amplified fragment to detect and identify the extent of microbial infestation present in the CSF biopsy for diagnostic detection purposes.

2. The method recited in claim 1 wherein the amplified fragment includes conserved sequence regions of a microbial genome of the nucleic acid, and wherein the manipulating step further comprises the steps of:
determining a nucleic acid sequence for the conserved regions of the amplified microbial genome; and
comparing each determined nucleic acid sequence with a database to identify the extent of microbial infestation present in the CSF biopsy.

3. The method recited in claim 2 further comprising the step of selecting a plurality of different primers for respectively separate interactions with different sequence regions of the microbial genome.

4. The method recited in claim 3 wherein the conserved regions in the amplified fragment include 16S, 23S, and Internal Transcribed Sequence (ITS) ribosomal regions of the microbial genome.

5. The method recited in claim 4 wherein a 1492 forward primer interacts with the 16S ribosomal region of the microbial genome, and a 457 reverse primer interacts with the 23S ribosomal region of the microbial genome.

6. The method recited in claim 2 wherein the determining step comprises the steps of:
cloning amplified fragments into a plasmid replication vector to create molecular clones; and
sequencing the molecular clones to identify the nucleic acid sequence.

7. The method recited in claim 2 wherein the determining step is accomplished by directly sequencing the amplified fragments to identify the nucleic acid sequence.

8. The method recited in claim 1 wherein the manipulating step further comprises the steps of:
capturing a plurality of amplified fragments on an array of separated oligonucleotides bound to a hybridization membrane; and
probing the amplified fragments on the array for positive hybridizations to evaluate the specified microbial infestation.

9. The method recited in claim 1 wherein the manipulating step further comprises the steps of:
providing a plurality of agar medium growth plates;
spreading a sample of the CSF biopsy on each plate; and
observing growth rates for the CSF sample on the different agar medium growth plates to identify a source of the microbial infestation.

10. The method recited in claim 1 wherein the amplifying step is accomplished using Polymerase Chain Reaction (PCR) amplification techniques.

11. The method recited in claim 1 further comprising the step of periodically repeating all steps of the method, as a testing procedure at predetermined time intervals, wherein the time intervals are less than two years.

12. The method recited in claim 1 wherein microbes are selected from the group consisting of bacteria, protozoa, fungi and archaea, and wherein the neurodegenerative disease is Alzheimer's disease.

13. The method recited in claim 1 wherein the nucleic acid is DNA.

14. The method recited in claim 13 wherein the DNA is created by reverse transcription of RNA.

15. An implementer for assaying microbes in the Cerebral Spinal Fluid (CSF) of a patient to detect an onset of a neurodegenerative disease which comprises:
a vial for holding a sample of the CSF, together with a plurality of beads and a liquid lysis buffer in the vial;
an oscillator for oscillating the vial and contents therein at a predetermined oscillation rate for a predetermined time duration, to break up host/microbial cells in the CSF for a release of nucleic acid therefrom and to dissolve the nucleic acid into solution with the lysis buffer, for subsequent incubation of the solubilized nucleic acid during a predetermined incubation period;
a pipette for removing solubilized nucleic acid from the vial for purification of the nucleic acid by extraction and precipitation;
a Polymerase Chain Reaction (PCR) amplifier for amplifying a selected fragment of the purified nucleic acid; and
a means for manipulating the amplified fragment to detect and identify the extent of a specified microbial infestation present in the CSF biopsy for diagnostic detection purposes.

16. The implementer recited in claim 15 wherein the amplified fragment includes conserved sequence regions of the microbial genome of the nucleic acid, and wherein the means for manipulating the amplified fragment comprises:
a computer for identifying the amplified fragment to establish a DNA sequence for the conserved regions of the amplified fragment of microbial genome; and
a comparator for comparing the identified DNA sequence with a DNA database to determine the extent of microbial infestation present in the CSF sample.

17. The implementer recited in claim 15 further comprising:
a kit for molecularly cloning the PCR DNA into a plasmid replication vector; and
an instrument for establishing a DNA sequence for the amplified fragments.

18. The implementer recited in claim 15 further comprising:
a hybridization membrane;
an array of separated oligonucleotides bound to the hybridization membrane; and
a labeled probe for detecting positive hybridizations on the membrane to evaluate the specified microbial infestation.

19. The implementer recited in claim 15 further comprising a plurality of agar medium growth plates for respectively receiving a portion of the CSF sample to determine a bacterial type for the CSF sample based on microbial growth activity.

20. The implementer recited in claim 15 wherein microbes are selected from the group consisting of bacteria, protozoa, fungi and archaea, and wherein the neurodegenerative disease is Alzheimer's disease.
